(19) United States
(12) Patent Application Publication
(10) Pub. No.: US 2008/0119847 A1
(43) Pub. Date: May 22, 2008

(54) BONE PEG FOR USE IN A BONE FIXATION SYSTEM

(75) Inventors: Jackson Heavener, Warsaw, IN (US); Shawn E. McGinley, Fort Wayne, IN (US); James S. Collins, Fort Wayne, IN (US); Donald M. Patmore, Winona Lake, IN (US)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER TECHNOLOGY, INC., Warsaw, IN (US)

(21) Appl. No.: 11/560,501

(22) Filed: Nov. 16, 2006

(51) Int. Cl. A61B 17/58

(52) U.S. Cl. 606/60; 606/75

(57) ABSTRACT

A bone peg which provides stable, accurate, and repeatable fixation with a bone fixation device to provide a system of fixation between a patient's anatomy, for example, a bone, and the bone fixation device.

### BACKGROUND

1. Field of the Invention

The present invention relates to a bone fixation system. More particularly, the present invention relates to a bone peg utilized to provide stable, accurate, and repeatable fixation with a bone fixation device.

2. Description of the Related Art

Conventional methods used to fixate a patient's anatomy, for example, a bone, and prevent movement thereof during a surgical procedure typically involve screws driven directly into the bone. These screws may be used to secure a bone fixation device to the bone. The screws used in such a manner typically extend into the cancellous portion of the bone. The bone fixation device is then secured to an operating table or other fixed structure in the operating room to provide a stable fixation construct which prevents movement of the bone during the procedure.

### SUMMARY

The present invention provides a bone peg which provides stable, accurate, and repeatable fixation with a bone fixation device to provide a system of fixation between a patient's anatomy, for example, a bone, and the bone fixation device. The fixation system advantageously prevents the bone from moving during a surgical procedure.

In one form thereof, the present invention provides a bone fixation system including a bone peg for implantation in a bone including an anchor portion, the anchor portion including a cannulated portion; and a head portion, the head portion including an aperture generally aligned with the cannulated portion; and a surgical instrument, the surgical instrument releasably engageable with the cannulated portion and with a support structure.

In another form thereof, the present invention provides a bone fixation system for fixating a bone including a bone fixation device; and fixation means for accurately coupling, decoupling, and recoupling the bone fixation device to the bone.

In yet another form thereof, the present invention provides a method for securing a bone to a bone fixation device including the steps of providing a bone peg, the bone peg including an anchor portion, the anchor portion including a cannulated portion; and a head portion, the head portion including an aperture generally aligned with the cannulated portion; implanting the bone peg in the bone; and releasably coupling the bone fixation device and the bone peg.

### BRIEF DESCRIPTION OF THE DRAWINGS

The above-mentioned and other features and advantages of this invention, and the manner of attaining them, will become more apparent and the invention itself will be better understood by reference to the following description of embodiments of the invention taken in conjunction with the accompanying drawings, wherein:

FIG. 1 is a perspective view of a bone peg in accordance with the present invention, further illustrating a bone fixation device, a bone peg insertion device, and a portion of a patient's anatomy including the knee joint;

FIG. 2 is a perspective view of the bone peg of FIG. 1;

FIG. 3 is another perspective view of the bone peg of FIG. 1;

FIG. 4 is a cross-sectional view of the bone peg of FIG. 2, taken along line 4-4 of FIG. 2; and

FIG. 5 is a perspective view of the bone peg insertion device of FIG. 1.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

The present invention generally provides a bone peg which is inserted or implanted in a patient's anatomy, for example, a bone. The bone peg provides stable, accurate, rigid, and repeatable fixation to a bone fixation device which fixates the bone relative to an operating table or other structure used in a surgical procedure. Advantageously, the bone fixation device may be coupled to the bone peg to permit fixation of the bone relative to the operating table. Furthermore, the bone peg also permits repeatable decoupling and recoupling of the bone fixation device to the bone peg throughout the surgical procedure without increasing the risk of potential damage to the bone, thereby removing the potential for decreased accuracy of the surgical procedure.

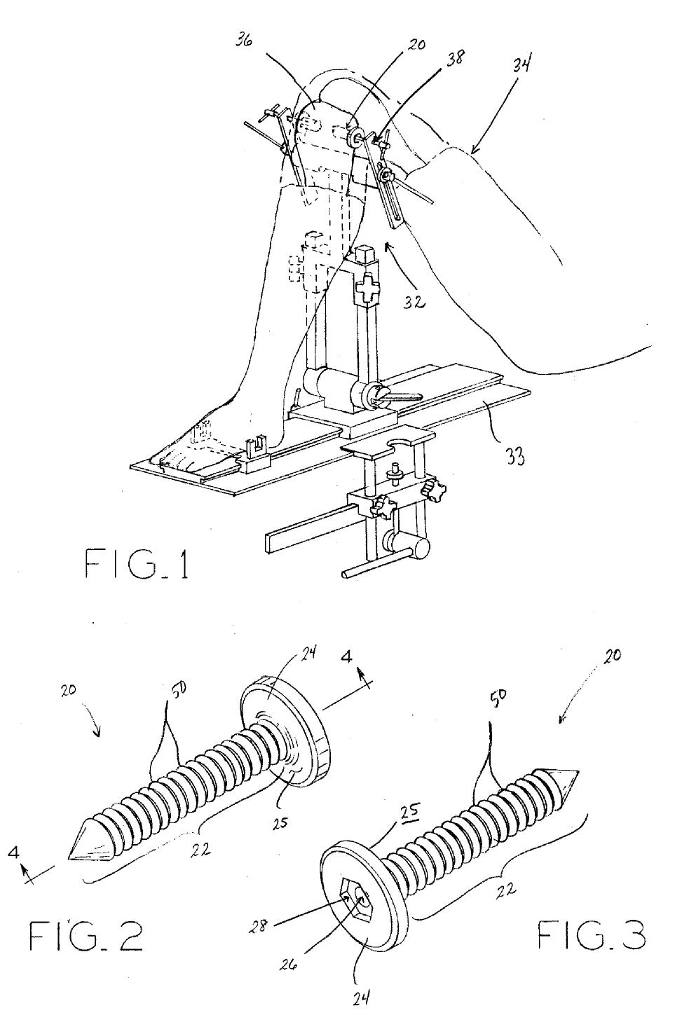

Referring now to FIG. 1, bone peg 20 may be used in a fixation system for stabilizing and securing tibia bone 36 of leg 34. Although shown in a system for fixating a tibia, bone peg 20 may be used with any other bone or anatomical structure. The system may include surgical instrument 32, such as a bone fixation device, for example, used to fixate tibia bone 36 relative to a support structure, such as operating table 33, for example. Surgical instrument 32 may also be a cut guide, a drill guide, an alignment device, a tracking array or device for use in a computer assisted surgery system, or any other surgical instrument that may require decoupling and recoupling to a bone of a patient during a surgical procedure. Bone fixation device 32 is secured to operating table 33 via clamps or other securing devices. Bone fixation device 32 may include bone peg insertion device 38, described below.

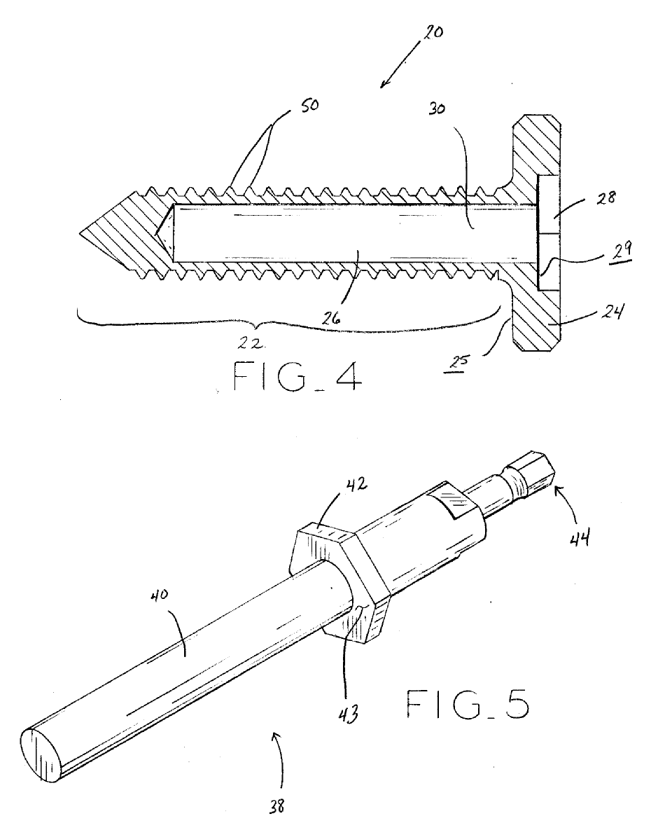

Referring now to FIGS. 2-4, bone peg 20 may include anchor portion 22 and head portion 24. Head portion 24 may include aperture 28 having a hexagonal cross-sectional shape and contact surface 25 having a substantially larger cross-sectional shape than anchor portion 22 when viewing bone peg 20 from either end. The relatively large size of contact surface 25 facilitates securement of bone peg 20 in tibia bone 36, as described below, and prevents movement of bone peg 20 in tibia bone 36 because contact surface 25 abuts the cortical bone surface of tibia bone 36 to provide a stable abutment therebetween. Anchor portion 22 may include cannulated or hollow portion 26 which extends substantially throughout the longitudinal length of anchor portion 22. As shown in FIG. 4, a central axis of aperture 28 is generally coaxial with a central axis of cannulated portion 26 to facilitate general alignment of aperture 28 with cannulated portion 26. Bone peg 20 may include threads 50 disposed on an exterior portion thereof to facilitate and enhance fixation of bone peg 20 within tibia bone 36 (FIG. 1).

In an alternative embodiment, bone peg 20 has no threads and may include a spike at a distal end thereof, similar to that shown in FIGS. 2-4, to facilitate driving bone peg 20 into tibia bone 36 via any suitable impaction device.

In an alternative embodiment, bone peg 20 has no threads and may include a substantially smooth exterior portion which engages with a pre-drilled hole in tibia bone 36 via an interference fit.

In yet another alternative embodiment, bone peg 20 has no threads and, upon engagement with a fixation instrument, for example, bone peg insertion device 38 or a fixation dowel or element of bone fixation device 32, is radially expanded to be secured in a pre-drilled hole in tibia bone 36. In this embodiment, bone peg 20 may include a radially expandable fixation portion. Several examples of radially expandable fixation devices are described in U.S. patent application Ser. No. 11/243,789, titled EXPANDABLE FIXATION DEVICES FOR MINIMALLY INVASIVE SURGERY, published on Mar. 9, 2006 as U.S. Patent Application Publication No. 2006/0052788, assigned to the assignee of the present application, the disclosure of which is expressly incorporated herein by reference.

Referring now to FIG. 4, cannulated portion 26 in anchor portion 22 and aperture 28 in head portion 24 together define cannula 30 which extends from head portion 24 substantially along the longitudinal length of anchor portion 22.

Referring now to FIG. 5, bone peg insertion device 38 may include elongated portion 40, head engaging portion 42, and tool engagement structure 44. Elongated portion 40 has a cross-sectional shape which substantially matches the cross-sectional shape of cannulated portion 26 of anchor portion 22 and head engaging portion 42 has a cross-sectional shape which substantially matches the cross-sectional shape of aperture 28 in head portion 24. Tool engagement structure 44 may include any well-known structure for engagement with a rotation-imparting tool or impaction tool (not shown). As shown in FIGS. 3 and 5, head engaging portion 42 and aperture 28 have a substantially hexagonal cross-sectional shape, thereby preventing any relative rotation between bone peg insertion device 38 and bone peg 20 when head engaging portion 42 is inserted in aperture 28. Prevention of relative rotation between bone peg insertion device 38 and bone peg 20 facilitates implanting bone peg 20 into tibia bone 36 via the rotation-imparting or impaction tool. Similarly, although shown as having circular cross-sectional shapes in FIG. 3 and 5, cannulated portion 26 and elongated portion 40 may have elliptical or polygonal cross-sectional shapes to further prevent relative rotational movement between bone peg 20 and insertion device 38. In an exemplary embodiment, elongated portion 40 has a cross-sectional shape which is compatible with a cross-sectional shape of cannulated portion 26.

Head engaging portion 42 includes bottom surface 43 which, when insertion device 38 is fully inserted into bone peg 20, abuts impaction surface 29 of aperture 28 to facilitate transfer of force from the rotation-imparting or impaction tool to bone peg 20. Furthermore, the abutting of bottom surface 43 and impaction surface 29 facilitates increased accuracy when decoupling and recoupling bone peg insertion device 38 and bone peg 20 because the surface abutment provides a repeatable landmark to the surgeon to facilitate returning bone fixation device 32 to the same location every time bone fixation device 32 is coupled to bone peg 20.

Bone peg 20 may be formed of any biocompatible material. In one embodiment, bone peg 20 may be made out of a bioresorbable material, thereby allowing bone peg 20 to be left in the body after the surgical procedure is completed. Alternatively, bone peg 20 could be made of a polymeric or metallic material. In a further alternative embodiment, bone peg 20 may be made of a radiopaque material which may facilitate determining relative implant position over time.

In operation and referring to FIG. 1, an exemplary surgical procedure involving the use of bone peg 20 generally includes an initial step of incising leg 34 proximate tibia bone 36 at a location near where bone peg 20 is to be implanted. After incising leg 34 and developing the wound to expose tibia bone 36, bone peg insertion device 38 may be inserted into bone peg 20 until bottom surface 43 of head engaging portion 42 abuts impaction surface 29 of aperture 28. Using the rotation-imparting or impaction tool, bone peg 20 is then driven into tibia bone 36 until contact surface 25 of head portion 24 abuts tibia bone 36. Bone peg insertion device 38 may remain in engagement with bone peg 20, and thereafter fixedly attached to bone fixation device 32, as shown in FIG. 1. Alternatively, bone peg insertion device 38 may be removed from engagement with bone peg 20 after which a fixation dowel associated with bone fixation device 32 is coupled to bone peg 20 to provide fixation of tibia bone 36.

Bone peg 20 may include a securement mechanism to secure the fixation dowel or bone peg insertion device 38 to bone peg 20. In one embodiment, the fixation dowel and/or bone peg insertion device 38 may include threads which engage a threaded cannulated portion 26 to provide fixation between bone fixation device 32 and bone peg 20. In yet another embodiment, the fixation dowel and/or bone peg insertion device 38 may include a groove configured to mate with a keyway provided in cannulated portion 26 to provide fixation between bone fixation device 32 and bone peg 20. In still another embodiment, the fixation dowel and/or bone peg insertion device 38 may include a retractable protrusion which mates with a groove or recess located within cannulated portion 26, e.g., a snap-fit engagement, to provide fixation between bone fixation device 32 and bone peg 20. In any of the embodiments described above for the securement mechanism, bone peg insertion device 38 may be formed without head engaging portion 42.

Advantageously, bone fixation device 32 is readily releasably coupled to bone peg 20 to provide stable fixation of tibia bone 36 relative to operating table 33. The readily releasable coupling permits bone fixation device 32 to be decoupled from tibia bone 36 without requiring removal of bone peg 20 from tibia bone 36 and without requiring substantial effort to decouple bone fixation device 32 from bone peg 20. Bone peg 20 provides a repeatable and accurate landmark where fixation between bone fixation device 32 and tibia bone 36 may repeatedly occur. Advantageously, eliminating the removal of bone peg 20 every time that bone fixation device 32 is decoupled from tibia bone 36 prevents potential damage to the cancellous portion of tibia bone 36 due to the repeated engagement of threads 50 with the cancellous bone. Moreover, bone peg 20 provides a repeatable and stable fixation location to facilitate better fixation and greater accuracy in repeatedly fixing tibia bone 36 relative to operating table 33. During a surgical procedure, a surgeon may advantageously decouple and recouple bone fixation device 32 with bone peg 20 in a predictable manner.

In an alternative embodiment, a plurality of bone pegs 20 may be used in an external fixation system. For example, two bone pegs 20 may be positioned in a bone, or multiple bones, at two separate locations and then connected with a rod or other connection device for use in a trauma application, for example.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A bone fixation system, comprising:
a bone peg for implantation in a bone comprising:
an anchor portion, said anchor portion including a cannulated portion; and
a head portion, said head portion including an aperture generally aligned with said cannulated portion; and
a surgical instrument, said surgical instrument releasably engageable with said cannulated portion and with a support structure.

2. The bone fixation system of claim 1, wherein said surgical instrument comprises a bone fixation device.

3. The bone fixation system of claim 1, wherein said surgical instrument includes a bone peg insertion device, said bone peg insertion device releasably engageable with said cannulated portion.

4. The bone fixation system of claim 1, wherein said surgical instrument includes a fixation dowel, said fixation dowel releasably engageable with said cannulated portion.

5. The bone fixation system of claim 1, wherein said head portion includes a contact surface, said contact surface abutting the bone when said bone peg is implanted therein.

6. The bone fixation system of claim 1, wherein said bone peg includes a securement mechanism.

7. The bone fixation system of claim 1, wherein said cannulated portion includes a cross-sectional shape, wherein said cross-sectional shape is substantially circular, substantially elliptical, or substantially polygonal.

8. The bone fixation system of claim 1, wherein said bone peg comprises a biocompatible material, wherein said biocompatible material is a bioresorbable material, a polymeric material, a metallic material, or a radiopaque material.

9. A bone fixation system for fixating a bone, comprising:
a bone fixation device; and
fixation means for accurately coupling, decoupling, and recoupling said bone fixation device to the bone.

10. The bone fixation system of claim 9, wherein said fixation means comprises a bone peg for implantation in the bone, said bone peg comprising:
anchoring means for anchoring said bone peg in the bone; and
coupling means for coupling said bone peg and said bone fixation device.

11. The bone fixation system of claim 10, wherein said coupling means comprises implantation means for implanting said anchoring means in the bone.

12. The bone fixation system of claim 10, wherein said bone peg comprises a biocompatible material, wherein said biocompatible material is a bioresorbable material, a polymeric material, a metallic material, or a radiopaque material.

13. The bone fixation system of claim 9, wherein said fixation means comprises a cannulated portion, said cannulated portion releasably engageable with said bone fixation device.

14. The bone fixation system of claim 13, wherein said cannulated portion includes a cross-sectional shape, wherein said cross-sectional shape is substantially circular, substantially elliptical, or substantially polygonal.

15. The bone fixation system of claim 9, further comprising implantation means for implanting said fixation means in the bone.

16. A method for securing a bone to a bone fixation device, comprising the steps of:
providing a bone peg, the bone peg comprising:
an anchor portion, the anchor portion including a cannulated portion; and
a head portion, the head portion including an aperture generally aligned with the cannulated portion;
implanting the bone peg in the bone; and
releasably coupling the bone fixation device and the bone peg.

17. The method of claim 16, further comprising the steps of decoupling the bone fixation device and the bone peg, and, subsequently, recoupling the bone fixation device and the bone peg.

18. The method of claim 16, wherein said implanting step comprises inserting a bone peg insertion device into the cannulated portion and driving the bone peg into the bone.

19. The method of claim 16, wherein said coupling step comprises inserting a bone peg insertion device into the cannulated portion and fixating the bone peg insertion device to the bone fixation device.

20. The method of claim 16, wherein the bone fixation device includes a fixation dowel, said coupling step comprising inserting the fixation dowel into the cannulated portion.
